(19) United States
(12) Patent Application Publication
(10) Pub. No.: US 2023/0041982 A1
(43) Pub. Date: Feb. 9, 2023

(54) SYSTEM AND METHOD FOR GENERATING A LIST OF PROBABILITIES ASSOCIATED WITH A LIST OF DISEASES, COMPUTER PROGRAM PRODUCT

(71) Applicant: E-DIAGMED, PARIS (FR)

(72) Inventors: Yannis Emmanuel Serge TSOUDEROS, NEUILLY SUR SEINE (FR); Nicolas GARNIER, SAINT MARTIN D'HÈRES (FR)

(21) Appl. No.: 17/966,366

(22) Filed: Oct. 14, 2022

Related U.S. Application Data
(63) Continuation of application No. 17/259,820, filed on Jan. 12, 2021.

(30) Foreign Application Priority Data
Jul. 13, 2018 (FR) 1800757

(51) Int. Cl. G16H 50/20; G16H 15/00; G16H 50/70; G16H 10/20; G16B 40/00; G16B 20/40

(52) U.S. Cl. CPC G16H 50/20; G16B 20/40; G16B 40/00; G16H 10/20; G16H 15/00; G16H 50/70

(57) ABSTRACT

A method for generating a list of probabilities associated with a list of diseases for a first patient, the method including first acquiring a first set of data of the first patient including an age value, a gender value; second acquiring data describing a disease of the patient, the disease being extracted from a first database, each disease being associated with a first prevalence statistic and a first incidence statistic, and each disease being associated with a list of signs; third acquiring data describing a first sign that includes a first sensitivity statistic and a second specificity statistic for each disease of a predefined list of diseases associated with the sign; generating, from a first modelling of a Bayesian network and input data including the data of the first, second and third acquisitions, of a set of probabilities, each probability being associated with a given disease of the first list.

### CROSS-REFERENCE TO RELATED APPLICATIONS

This application is a continuation of application serial no. 17/259,820, filed on Jan. 12, 2021, which is the U.S. National Stage of PCT/EP2019/068848, filed Jul. 12, 2019, which in turn claims priority to French patent application number 1800757 filed Jul. 13, 2018. The content of these applications are incorporated herein by reference in their entireties

### FIELD

The field of the invention relates to systems and methods, notably methods implemented by computer, making it possible to generate a list of conditional probabilities which are associated with diseases. The field of the invention aims to provide a tool, or even a simulator, based on a modelling of a Bayesian network enabling a user to established diagnoses on the basis of formalized hypotheses. The field of the invention also relates to methods aiming to detect the influence of a factor in the description of a disease or in a particular and identified clinical form of this disease.

### PRIOR ART

At present, numerous methods exist making it possible to assist a physician in establishing a diagnosis for a patient. Generally, these methods establish a data model making it possible to associate the appearance of a factor with the onset of a disease, the association being able to be made, for example, by the modelling and the calculation of a probability. The physician may then define a certain number of input factors such as signs, also called symptoms in medical terminology, on order to know the set of diseases being able to induce this symptom.

One of the models being able to be implemented is a Bayesian type model. Such a data model makes it possible to generate conditional probabilities as a function of a certain number of factors identified in a patient. An interest is to establish a tool making it possible to help a user or a physician to establish diagnoses. This is for example the case of the solution described in the patent document US7720779 in which a logical influential relationship is modelled.

A problem is that logical influential relationships can lead to excluding possibilities or taking into account a too large database of potential diseases. This modelling may lead a user to commit errors in establishing his diagnosis. One reason is that the signs observed may differ from one individual to another, from one clinical form to the other of a same disease and from one disease to the other. Finally, modelling of a probability based uniquely on a logical relationship does not make it possible to generate relevant lists of probabilities associated with diseases. Indeed, the logical relationship does not model, for example, a level of specificity of a sign in a given list of diseases being able to induce it.

There exists a need to propose a device, and a method, making it possible to provide an assistance tool to a user so that he can make reliable diagnoses.

### SUMMARY

The method of the invention makes it possible to resolve the aforesaid problems.

According to an aspect, the invention relates to a system comprising a calculator for generating a list of probabilities associated with a list of diseases for a first patient, said system comprising an interface enabling:
a first acquisition of a first set of first factors relative to the first patient and storing said first factors in a memory, said first factors comprising:
   an age value,
   a gender value;
a second acquisition of a second set of second factors, called risk factors, notably describing at least one disease or information specific to said patient and storing said second factors in a memory, said disease being extracted from a first database, each disease being associated with a first prevalence statistic and/or a first incidence statistic, and each disease being associated with a list of signs;
a third acquisition of a third set of third factors describing at least one sign and storing said third factors in a memory of the system, said first sign comprising a first sensitivity statistic and a second specificity statistic for each disease of a predefined list of diseases associated with said sign;

said calculator generating, from a first modelling of a Bayesian network and input data comprising the data of the first, second and third acquisitions, a set of probabilities, each probability being associated with a given disease of the first list; said system comprising a graphic interface to display said generated first list.

An advantage is to enable calculations of probabilities associated with diseases to be made reliable thanks to a model integrating a codification of the sensitivity and specificity of factors. Another interest is to consider different types of factors, such as the profile of a user, that is to say of a patient, the medical histories and the symptoms of said patient, in the evaluation of the probabilities associated with diseases.

Moreover, an advantage is to construct nodes of a Bayesian network to optimize the probability calculations as a function of a plurality of factors.

According to an embodiment, the system comprises an interface enabling a fourth acquisition of fourth factors describing a given medical product and at least one second sign associated with said given medical product, said second sign comprising a first sensitivity statistic and a second specificity statistic for each disease of a second predefined list of diseases associated with said second sign; the calculator generating the set of probabilities while taking into account as input the data of the fourth acquisition to generate the first list.

An advantage is to take into account in the calculation of probabilities associated with each disease data relative to an active principle capable of interfering in the estimation of probabilities.

According to an embodiment, the system comprises a memory for saving data describing at least two medical products and saving data coming from a plurality of data acquisitions derived from a set of patients in such a way that a first group of patients is associated with the first product and a second group of patients is associated with the second product, the calculator generating two lists of conditional probabilities, each probability being associated with a given disease, the calculator performing a calculation to compare the two lists in order to deduce the presence of at least one difference in probabilities for a same disease between the two lists when said difference is above a predefined threshold.

An advantage is to identify unknown pharmacological effects of a given active principle when it is tested in comparison with another active principle or a placebo.

According to an embodiment, the system comprises a linguistic resource comprising an ontology, a dictionary or a synonyms database making it possible to associate terms describing signs or diseases in the database with a predefined textual corpus.

An advantage is to homogenize the description of signs in order to normalize the sensitivity and specificity codifications of the signs. Moreover, another advantage is to take into consideration descriptions made by patients and translated automatically. Another interest is to suggest choices automatically through the interface in order to better describe a sign when a detailed sign is associated with a sensitivity or with a predefined specificity in a diseases database. It is then possible to generate proposals automatically in the interface to complete the description of a sign as a function of the probabilities calculated and associated with diseases. Consequently, the user is helped to better define existing signs or symptoms.

According to an embodiment, each third factor comprises a plurality of properties describing a sign and stored in a memory of the system, each property being associated with a sensitivity and specificity value.

According to an embodiment, the first modelling of the Bayesian network comprises, for at least one node of the network, a modelling of at least one relationship between two factors of one of four sets of factors, said modelling specifying if the factors are independent or dependent and being stored in a memory of the system.

An advantage is to model signs having a relationship with each other differently from signs not having any relationship with each other or for which the relationship is not known. Thus, the invention makes it possible to model nodes of the Bayesian network according to different levels of knowledge of the causal relationships between signs.

According to an embodiment:
when no dependency relationship between at least two factors present in the acquired data is specified, the calculation of the probability of a disease comprises a calculation of conditional probability from factors considered as independent;
when said Bayesian modelling specifies a dependency relationship between at least two factors, the probability of a disease comprises a selection in the database either of the joint conditional probability or a selection of a sensitivity and/or specificity value of factors considered jointly.

An advantage is to model a Bayesian network that is the most faithful possible to reality. Thus, the modelling of nodes takes into account the existence of certain types of relationships between signs when it exists. In the opposite case, the calculations of probabilities are performed by considering the factors as independent.

According to an embodiment, the system comprises a calculatorto establish a comparison between data acquired through the interface and data stored in the memory such that when an acquired risk factor is associated with a prevalence or incidence statistic of a disease, the probability of the associated disease is initialized at the value stored in the database.

According to another aspect, the invention relates to a method for generating a list of probabilities associated with a list of diseases for a first patient, said method comprising:
First acquisition of a first set of first factors relative to the first patient comprising:
   o an age value,
   o a gender value;
Second acquisition of a second set of second factors, called risk factors, notably describing at least one disease or information specific to said patient, said disease being extracted from a first database, each disease being associated with a first prevalence statistic and a first incidence statistic, and each disease being associated with a list of signs;
Third acquisition of a third set of third factors describing at least one sign, said first sign comprising a first sensitivity statistic and a second specificity statistic for each disease of a predefined list of diseases associated with said sign;
Application of a first modelling of a Bayesian network to input data comprising the data of the first, second and third acquisitions, for the generation of a set of probabilities, each probability being associated with a given disease of the first list.

An advantage is to implement the method in such a way that it can access a database of signs and/or diseases so as to calculate from data collected by an interface a list of diseases, each being associated with a probability. An advantage is to take into account a given profile of a user and data specific to this user to make the probability data that are generated reliable.

According to an embodiment, the probability associated with a disease of the first list is a conditional probability of a disease with the occurrence of a set of factors comprising at least three elements among the following list:
a predefined sign;
a predefined medical history;
a risk factor;
a predefined age;
a predefined gender;
a predefined geographic location;

An advantage is to take into account numerous factors for improving the precision of the probabilities which are generated in each list.

According to an embodiment, at least one factor of the second acquisition of data is associated with a medical history and comprises at least one of the following criteria:
a first date of appearance;
a frequency of appearance;
a genetic information.

An advantage is to select, if need be, a prevalence or incidence statistic in the database of signs or diseases that is specific to a second factor corresponding to a medical history qualified by at least one additional datum. Thus, the database of signs or diseases comprises a plurality of statistics assigned to different qualifications of medical histories.

According to an embodiment, the first modelling of the Bayesian network comprises, for at least one node of the network, a modelling of at least one relationship between two factors of one of the four sets of factors, said modelling specifying if the factors are independent or dependent, the dependency relationship between at least two factors being modelled by a value of joint conditional probability of a disease knowing the factors presents.

According to an embodiment, the method comprises a step of verifying the existence of a relationship between factors acquired from the different sets in the database of signs or diseases, if need be, the method comprises a step of selecting the value of the joint probability associated with the linked factors.

An advantage is to model a Bayesian network taking account of a level of detail of the database of signs or diseases. The level of detail is defined by the definition of prevalence or incidence statistics or specificity of signs for a disease and of sensitivity of signs in a disease considered jointly with other factors. When a new statistic, or probability, is defined, the Bayesian network is modelled in such a way that its nodes take into account the relationships between factors affected by this new statistic. Thus, according to the data acquired of a user, the calculations of probabilities are improved.

According to an embodiment, the method comprises a fourth acquisition of fourth factors describing a given medical product and at least one second sign associated with said given medical product, said second sign comprising a first sensitivity statistic and a second specificity statistic for each disease of a second predefined list of diseases associated with said second sign; the step of generating the set of probabilities taking into account as input the data of the fourth acquisition.

An advantage is to make it possible to identify the probabilities associated with diseases being able to be assigned notably to the presence of a fourth factor.

According to an embodiment, the sensitivity or the specificity of a sign of an input of the first database comprises:
either a value expressing a probability;
or a value selected from a predefined discrete scale, said predefined discrete scale associating with each of its values a probability by age and/or gender group.

An advantage is to normalize the sensitivity and specificity values so as to obtain homogeneous calculations and being able to be quantified by an “expert’s opinion”. Thus, an expert’s opinion could quantify the sensitivity on a scale, for example comprising between 5 and 8 levels to quantify the values, whereas he could not quantify precisely a statistic to an exact percentage, for example sensitivity of a sign. Thus, this modelling makes it possible to acquire information having a granularity which can be given by an expert.

According to an embodiment, the method is carried out for a plurality of patients, the method comprising a plurality of data acquisitions in such a way that each patient is associated with a first product or with a second product, a first group of patients being associated with the first product and a second group of patients being associated with the second product, the generation step comprising the generation of two lists of conditional probabilities, each probability being associated with a given disease, the method comprising, moreover, a step of comparing the two lists in order to deduce therefrom the presence of at least one difference in probabilities associated with a same disease and for which the difference is above a predefined threshold.

An advantage is to make it possible to use the invention for pharmacovigilance applications.

According to an embodiment, the third acquisition is carried out by means of an interface in which the selection of a given sign automatically leads to the generation of a first selection of signs, said selected signs being associated with the given sign in at least one disease, said interface comprising a menu displaying said selection of signs.

An advantage is to benefit from relationships established between the different factors when they exist. This makes it possible to suggest automatically by means of the interface choices in the definition of the data input by a user. The choices proposed are those which are the most capable of being verified by the presence and the quantification of relationships between factors. To do so, the relationships between factors that are modelled by a statistic/probability may be proposed, to a user, according to a certain order according to the values of probabilities.

According to an embodiment, the third acquisition leads to the generation of a second selection of tests, said tests comprising a description aiming to identify the presence of at least one sign in the patient.

According to another aspect, the invention relates to a computer program product loadable directly in the internal memory of a digital computer, comprising software code portions for the execution of the steps of the method of the invention when said program is executed on a computer.

According to another aspect, the invention relates to a computer program product stored on a support that may be used in a computer, comprising at least one calculator and a memory in order to execute a command for the implementation of the method of the invention.

### BRIEF DESCRIPTION OF THE FIGURES

Other characteristics and advantages of the invention will become clear on reading the detailed description that follows, with reference to the appended figures, which illustrate:

FIG. 1: a general architecture for the implementation of the method of the invention and /or the system of the invention;

FIG. 2: an ontology of signs according to their manifestation and their description;

FIG. 3: a diagram of the different factors being taken into account in the calculation of a probability of the onset of a disease according to an embodiment of the system of the invention.

### DESCRIPTION

### Definitions

The sensitivity of a test measures its capacity to provide a positive result when a hypothesis is verified. In the case of the invention, the sensitivity of a sign measures its possibility of being present in the manifestation of a disease. Sensitivity may be expressed in percentage or in ratio. According to an embodiment of the invention, the level of details qualifying the description of a sign makes it possible to improve the relevance of the sensitivity of said sign.

The specificity measures the capacity of a test to give a negative result when the hypothesis is not verified. Within the scope of the invention, the specificity of a sign is its capacity to predict the non-presence of the disease when it is not present. It may also be expressed as the measurement of the specific character of a sign with respect to a disease. It may be expressed in percentage, that is to say a measurement indicating 90% specificity of a sign for a disease indicates a strong characterization of the presence of the disease when the sign is detected. According to an embodiment of the invention, the entering of a detailed and precise description of a symptom/sign makes it possible to modify the specificity value. The level of precision and detail of a sign may be associated with a scale of values of the specificity of a sign for a disease.

A sign may have low sensitivity in a disease and high specificity for the same disease, or vice versa, high sensitivity and little specificity.

An association of signs may have lower sensitivity in a disease and higher specificity for the same disease, respectively, than those for each of these isolated signs.

The prevalence PRi is a measurement of the state of health of a population, counting the number or proportion of cases of a disease at a given time or over a given period. Prevalence may be associated with a medical history or in other words with a risk factor. Prevalence may also be associated with a given demography. The taking into account in the definition of the demography of a section of age, gender, family and/or personal medical histories, or of a geographical zone or of a combination of these criteria may be associated with a given prevalence. Within the scope of the invention, a prevalence model comprises the different prevalence values over a given demographic segmentation and/or of risk factors.

The incidence INi of a disease measures the number of cases appearing over a predefined duration, for example a year, within a population. The incidence of a disease may be associated with a given demography. The taking into account in the definition of the demography of a section of age, gender or geographic zone or a combination of these criteria may be associated with a given incidence of the disease. Within the scope of the invention, an incidence model of a disease comprises the different incidence values of the disease over a given demographic segmentation and/or of risk factors.

A disease Mi is an alteration or a disorder of the body. Within the scope of the invention, a database of diseases BD_{M} is used. The database of diseases BD_{M} comprises fields of which the values characterize the disease and fields making it possible to define contextual indicators relative to its occurrence, such as its incidence or its prevalence.

A symptom Sᵢ is a clinical sign that results from a manifestation of a disease, such as expressed and felt by a patient. In the invention, each sign is described in a database. A symptom corresponds to the description that a patient gives of a sign. Thus, the terms and the ontology may differ between the description made by a patient and that of the database of signs.

Within the scope of the invention, a database of signs BDs is thus defined. According to an embodiment, a dictionary, a synonyms database or an ontology may be integrated in the system and in the method of the invention. A consequence is to make it possible to detect a sign or a property of a sign when certain terms are input in the interface by a user. Thus, a user may be guided by suggestions, definitions or explanations making it possible to interpret the description of a symptom to associate the properties of a sign therewith.

A given disease may have clinical tables different at time T compared to time T+dt and/or from one patient Pᵢ to the other patient Pₖ. The database of signs BDs comprises fields of which the values characterize said sign. Moreover, it comprises fields making it possible to define contextual indicators relative to the relationships between the sign and diseases or instead relationships between the signs themselves. These relationships make it possible to take into account weighting of values of factors defining input data in the calculation of a probability for a given disease. For each sign Sₖ, a list of diseases LIST_{Sk} is associated with, for each of them, a specificity value and a sensitivity value.

The database of diseases BD_{M} and that of signs BDs are thus linked notably by the specificities SPi for one or more disease(s) and the sensitivities SEi of signs in one or more diseases.

The database of diseases BD_{M} and/or that of signs BDs may thus be completed by a field relative to at least one sign Sᵢ associated with a given disease with the sensitivity and specificity values of said sign Sᵢ. In describing a symptom of a patient, it is then possible to select a sign Sₖ already entered in the database. An interface then makes it possible for a user to determine the characteristics of the symptom in order to filter the possible results present in the database.

A “medical history” is for example a disease which has been or which is declared by a patient. More generally, within the scope of the present invention, a medical history is a fact specific to a patient inducing a disease risk factor. As an example, a previous disease, a status of smoker, cholesterol above a given threshold, a genetic mutation, taking medication are facts attached to a patient which may be considered as risk factors for a set of diseases.

It corresponds to a disease contracted by the patient. The invention makes it possible to take into consideration in the profile of a patient a set of factors, of which the medical histories. The latter may affect the probabilities associated with a disease in so far as the latter may be for example specific of a disease.

This may be the case, for example, in taking into account the medical history: “Cirrhosis of the liver” during the calculation of the probability of the disease: “Hepatic encephalopathy”. Indeed, the medical history “Cirrhosis of the liver” here affects in a consequent manner the probability of the disease “Hepatic encephalopathy” due to the fact:
of the sensitivity value of “Cirrhosis of the liver” in “Hepatic encephalopathy” and;
of the specificity value of “Cirrhosis of the liver” for “Hepatic encephalopathy”.

### Modelling of the Sensitivity

According to an embodiment, a field of sensitivity of a sign relative to a disease is encoded according to a predefined scale of values, for example of 0 to 6. “0” corresponding to at the most 5% in sensitivity of a disease and “6” to at least 95% in sensitivity of a disease, between 1 and 5, the quotients or the percentages of sensitivity to a disease are determined according to a distribution of probabilities that is predefined. The distribution may be, for example, of Gaussian or linear type. Any other association curve is compatible with the invention. A linear model may, for example, be implemented. According to another example, the scale of values is established between 0 and 10. The invention is compatible with any other implementation of scale of values.

When the value of the sensitivity of a sign in a disease is known for an age, a gender, or a medical history or a given external pathogenic factor, the method of the invention makes it possible to take into account the value known in the database BD_{M} or BDS. This datum may make it possible to reinforce a model of distribution of sensitivity values for a given population.

In the same way, the sensitivity of a medical history in a disease is stored in the database of the system of the invention. According to an embodiment of the invention, the acquisition of a medical history ANT1 described in the profile of a patient P1 is taken into account in the determination of the probability of a disease P(M) when the value of the probability of the disease associated with the medical history is specified in the database. The invention thus makes it possible to model the relationships between medical histories and diseases by the definition of a set of probabilities. This probability may be interpreted as the sensitivity of a medical history in a disease.

### Modelling of the Specificity

According to an embodiment, a field of specificity of a sign Sₖ for a disease is encoded according to a predefined scale of values, for example from 0 to 6. According to another example, the scale of values is established between 0 and 10. The invention is compatible with any other implementation of scale of values. In an analogous manner to the modelling of the sensitivity, the values of the scale chosen to model the specificity may be based on a discrete distribution of probabilities for a given population. For example, if the scale is comprised between 0 and 6, “0” corresponding at the most to 5% in specificity of a sign for a disease and “6” at least to 95% in specificity of a sign for a disease. Between 1 and 5, the percentages of specificities are associated with the values of the scale according to a distribution of probabilities for a given population. The distribution may be, for example, of Gaussian or linear type. Any other association curve is compatible in the invention.

When the specificity value is known, the method of the invention makes it possible to take into account the value known in the database BD_{M}. In the event of conflict between a predefined scale and a given value, the method of the invention takes into account the known value stored in the database. An alarm may be generated during the detection of a case of conflict in order to attract the attention of the user or another person to the modelling of the specificity of a sign of a given disease.

In the same way, the specificity of the presence of a medical history for a disease is stored in the database of the system of the invention. From a point of view of the invention, the precision of a medical history specified in the profile of a patient acts in the same way as precision of a medical history. The invention thus makes it possible to model the relationships between medical histories and diseases by the definition of a set of probabilities of the specificity of a medical history relative to at least one disease.

### Detailed Sign

A “detailed sign” is also called a “qualified sign”.

The method comprises an automatic means for calculating the specificity value of a “detailed sign” for one or more diseases as a function of the data entered in the different fields of its description. It is recalled that a “detailed sign” comprises more information than a “generic sign”. This automatic calculation may, for example, be based on an abacus or a predefined scale making it possible to quantify the level of precision of the description of a sign and the specificity and sensitivity values that are associated with each gradient of said scale or of the predefined abacus.

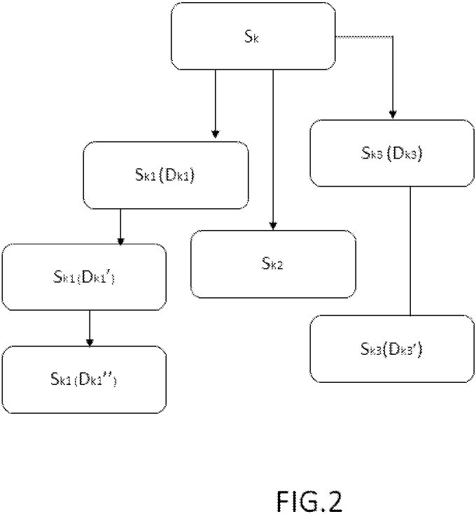

FIG. 2 represents a generic sign Sₖ and different manifestations of this sign, noted Sₖ₁, Sₖ₂, Sₖ₃. The sign Sₖ₁ is declined according to more or less detailed descriptions: Dₖ₁, Dₖ₁’, Dₖ₁”. In this example, the description Dₖ₁” is more detailed than the description Dₖ₁' which is, itself, more detailed than the description Dₖ₁. The reason for these differences may arise from a different acquisition of data or nuances in the manifestations of these signs according to the patients and even more according to the diseases.

According to an embodiment, the specificity SPₖ is encoded on a predefined scale which makes it possible to generate a percentage associated with the corresponding value during the modelling of the Bayesian network RB implemented.

The specificity value SPₖ may be predefined for each of the declinations/ manifestations of a sign Sₖ and for each type of description Dₖᵢ, Dₖᵢ’, Dₖᵢ” of the latter for one or more disease(s). The values are stored in a memory.

According to an embodiment, the specificity SPₖ of a “detailed sign” Sₖᵢ for a given disease may be obtained by considering a general specificity of the sign and by applying a weighting coefficient obtained as a function of the number of fields of the description. It then involves a quantitative weighting of which the principle is based on the fact that the more a sign Sₖ is detailed the more it is specific.

According to another example which may be combined with the latter, the value of the fields makes it possible to generate a weighting coefficient of the specificity of a sign Sₖ for one or more disease(s). In this case, it involves a qualitative weighting of which the principle is based on taking into account values specifying a description of a sign Sₖ.

According to an embodiment, the sensitivity value SEₖ of a sign Sₖ for a given disease M₁ is encoded in the same way as the specificity SPₖ. The same is true for a “detailed sensitivity” or a “generic sensitivity”.

Each sign Sₖ comprises a specificity value for one or more disease(s) which may be adjusted according to the detailed description Sₖᵢ of a manifestation of this sign. When the description of a sign Sₖᵢ is modified, according to an embodiment of the invention, an interface is generated enabling a user to modify the specificity values. By default, without modification by the user, the specificity value remains unchanged in the database.

According to an example, the specificity and sensitivity values of the following signs are entered in the database:
the sign: “pain” comprises a specificity of Sp = 0.0% and a sensitivity Se = 100%.
the sign qualified: “thoracic pain” comprises a specificity of Sp = x₁ > 0.0% and a sensitivity Se = y₁ < 100%.
the sign qualified “anterior pain” comprises a specificity of Sp= x₂ > x₁ and a sensitivity Se = y₂ < y₁.
the sign qualified “anterior thoracic pain” comprises a specificity of Sp = x₃ > x₁ and a sensitivity Se = y₃ < y₁.

The sensitivity Se and the specificity Sp are defined and modified for example by a user or several users having specific rights enabling them to access these data and to modify said data in the database, they are called “administrators”. According to an embodiment, for a user using the method or the system of the invention, the sensitivity and specificity values are determined and fixed during the use of the software implementing the method or the system of the invention.

According to an embodiment, the values are modified for example from an updating operated by an administrator of the database(s). According to another embodiment, the databases of the system may be automatically updated with a database centralizing the up to date values of the different parameters.

It is recalled that a more detailed sign Sᵢ will be more precise and thus less frequent, but furthermore more specific.

An interest of a modelling of a detailed sign is to associate with each characteristic of the sign a sensitivity and specificity value when the latter are known.

### Modelling of a Sign

According to an embodiment, an interface makes it possible to detail the description Dₖᵢ of a detailed sign Sₖᵢ, called “detailed sign”, of a patient. The detailed sign Sₖᵢ comprises at least the description of a generic sign Sₖ itself comprising at least one designation of the sign, for example: “fever”.

Moreover, according to different examples, the description of a detailed sign comprises different fields such as:
an intensity of the sign, for example defined on a predefined scale or according to predefined qualifiers;
a curve of occurrence of the sign, for example, reproducing a number of manifestations of the sign or an evolution of its occurrence;
a qualification of the sign such as, for example, a dry cough or a wet cough;
a value reproducing the persistence and the evolution of the sign, spontaneously or in the course of a treatment or after a change of posology or the taking of medication;
a context of manifestation of the sign, for example as a function of the time, the day or instead as a function of a climatic or geographic condition;
a medical examination result (radiological test, scanner, biology, palpations, etc.);
etc.

The more a description Dₖᵢ of a detailed sign Sₖᵢ is entered in the database BD_{M} or the database DBs, the more it makes it possible to obtain high probabilities for a given disease or a given set of diseases.

Indeed, each property of a sign may be modelled by a specificity and sensitivity value.

For each sign described, the invention makes it possible to qualify therefrom precisely the description by means of an enriched interface. A menu makes it possible for this sign to choose from among the different qualifiers that are possibly linked to it in the different diseases where it may exist. During a new input, for example when a new patient describes a sign for the first time, the method of the invention makes it possible: either to enrich an existing detailed sign, or to define a detailed sign. In the latter case, a menu makes it possible to select an existing sign and to edit a new version thereof in order to complete the latter with additional information collected, for example, by a user.

Here is presented in a table an example of modelling coming from an extract of a description of signs linked to confusion in the disease hepatic encephalopathy.

| Signs | Qualifiers | Sensitivity | Specificity |
|---|---|---|---|
| Onset | | | |
| | Very slow | 00 | 0 |
| | Rapid | 4 | 3 |
| | Sudden | 2 | 3 |
| Disorders | Fixed | 00 | 0 |
| | Fluctuating | 6 | 5 |

The values, here indicated on a scale of 1 to 6, make it possible to encode a fraction/a percentage which makes it possible to calculate a conditional probability of a disease when the sign is present. The sensitivity code 00 makes it possible to differentiate the disease when the code is present, that is to say when the sign or the detailed sign is present. For example, this may be the case during a normal examination result which in fact eliminates the possibility of a given disease.

In this example, the onset of the sign may be associated with a specificity value for a given disease and/or a sensitivity value in a disease. In an analogous manner, the modelling of disorders and their occurrence comprises the association of a specificity and sensitivity value to each attribute present in the model, here: “fixed” and “fluctuating”.

A same generic sign Sₖ may be associated with a list of different diseases Mₚ,_{pε[1},_{N]}. This generic sign Sₖ, may comprise different presentations Sₖ₁, Sₖ₂, Sₖᵢ, etc., for different diseases of the list of diseases Mₚ,_{pε[1},_{N]}. The characterization of each detailed sign is associated with a disease and reinforces the specificity of the detailed signs Ski. The invention thus makes it possible to attach a common concept, that is to say the designation of the sign, to different diseases while differentiating the fields characterizing the manifestations of the sign for each among them, that is to say the detailed signs.

In the same way, each disease Mₚ is associated with a list of signs Sₖᵢ in such a way that the databases of signs BDs and diseases BD_{M} are associated with each other.

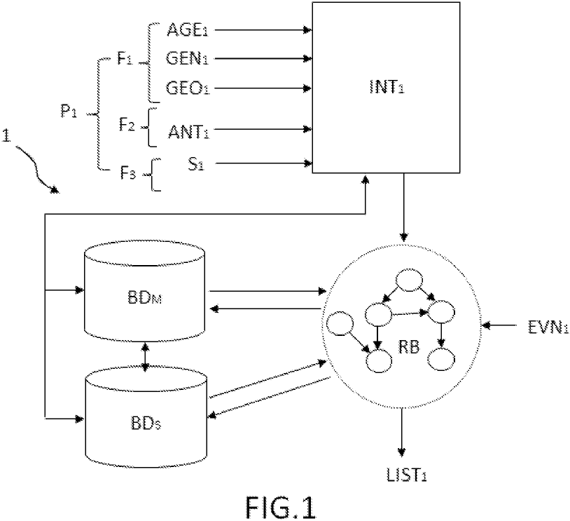

FIG. 1 represents an embodiment of the invention. A user interface INT₁ makes it possible to access functionalities implemented by the execution of the method of the invention. The interface INT₁ may be accessible from a smartphone, a computer or a digital tablet or instead any other machine comprising a memory and a calculator. It comprises menus, such as drop-down menus, and input fields making it possible to define or select information. Advantageously, the interface INT₁ offers a means of acquiring data.

Moreover, the interface INT₁ makes it possible to access a remote server or a local memory comprising a database of diseases BD_{M} and/or a database of signs BDs.

The method of the invention comprises a first step of acquisition of data ACQ₁ of a patient P₁. The data comprise an identifier in order to identify the patient P₁ in a unique manner. Moreover, the data defining the patient P₁ may comprise information designating said patient, such as an identifier or an encrypted datum being able to be decoded by an encryption/decryption means. According to an embodiment, data identified as confidential are encrypted.

### Generation of Test/Examination

The manner of characterizing a sign may be achieved in three successive phases comprising different steps carried out with a patient P₁:
description of a sign described by the patient (symptom);
carrying out clinical examination(s),
carryout out complementary examination(s), for example biological or imaging examinations.

The method of the invention makes it possible to generate automatically, at the end of the generation of a list of probabilities, an indicator mentioning a type of test to carry out and optionally its nature. According to an embodiment, when two probabilities associated with distinct diseases are substantially similar or when one of them is above a predefined threshold, a test is automatically proposed, whatever the value of the others.

As an example, if a first list LIST₁ comprises three diseases M₁, M₂, M₃ each associated with the following probabilities: 30%, 25%, 2%, it is then necessary to determine a specific test for the diseases M₁ and M₂. The invention then makes it possible to identify automatically a sign specific to one of the two diseases M₁ or M₂ and which is discriminating for each of the two diseases M₁ and M₂. The invention then makes it possible to generate automatically an indicator specifying a test or an examination making it possible to discriminate M₁ and M₂ according to the presence or not of the sign that an interface of the system of the invention suggests entering. For this purpose a database of tests/examinations may associate test protocols with signs and/or diseases. In this example, the difference between 25% and 30% is below a predefined threshold, for example 8%. With this first condition, the method comprises a step aiming to identify, for each of these diseases, at least one sign of which the specificity is below a certain threshold for one of the two diseases and above a certain other threshold for the other disease. The test making it possible to determine and verify the presence of the sign will then help a user to conclude in a robust diagnosis.

According to another example, if a first list LIST₁ comprises three diseases M₁, M₂, M₃ each associated with the following probabilities: 20%, 3%, 90%, it is then necessary to determine a specific test for the diseases M₁ and M₃. Even if the value of the probability of M₃ is very high, the invention makes it possible to recommend automatically the carrying out of an additional test to differentiate the possibility of M₁. The system of the invention comprises a database of referenced and described tests, each test being associated with a specificity for one or more disease(s). Thus, each of the diseases presented in the list LIST1 may be associated with one or more tests making it possible to improve the calculation of the probabilities of at least one disease of the list LIST₁.

The invention then makes it possible to identify automatically a sign specific to one or to the other of the diseases M₁ or M₃ and which is discriminating for each of the two diseases M₁ and M₃. The invention then makes it possible to generate automatically an indicator specifying a test or an examination making it possible to discriminate the presence or not of the sign. For this purpose, a database of tests/examinations may associate test protocols with signs and/or diseases. In this example, the threshold of 15% is overstepped by two diseases: M₁ and M₃. In this second condition, the method comprises a step aiming to identify a sign of which the specificity is below a certain threshold, or even critical, for one of the two diseases and above a certain other threshold for the other disease. The test making it possible to determine the presence of the sign will then aid a user to obtain more reliable probabilities in the list LIST₁.

### First Factors F1: The Data of Patients

The data acquired during the acquisition ACQ₁ are designated “first factors F₁”. They comprise at least an age AGE₁ and a gender GEN₁. This information is notably taken into account to select automatically a prevalence (or an annual incidence) of a disease associated with the profile of the patient P₁. For this purpose, the prevalence is determined from a prevalence model according to a given population in relation to the profile of the patient, of which the gender and the age. For example, the prevalence values may be distributed according to age or gender classes. According to an example, a menu proposes predefined fields making it possible to select information specific to the patient P1 in order to generate automatically the data of the model that will be associated therewith.

According to an embodiment, the data acquired of a patient P₁ comprise a geographic information GEO₁, for example a country, a region or a town/city. This data may also be taken into account in the determination of a prevalence of a disease. The prevalence values of a disease may be distributed according to geographic zones. According to an embodiment, an input zone makes it possible to define the geographic information. According to another embodiment, the geographic information GEO₁ is selected from a menu making it possible to extract geographic data that have been predefined in a local or remote memory.

According to an embodiment, the interface INT₁ makes it possible to select at least one medical history ANT₁, that is to say a disease M₁ of a patient P₁ that has occurred at a previous or current date. These data are acquired during an data acquisition ACQ₂. This acquisition ACQ₂ may be carried out from the same interface INT₁ as the interface having enabled the acquisition of the first factors F₁. According to another embodiment, a second interface INT₂ may be used, for example, succeeding the first interface INT₁ after having validated the data acquired by this first interface INT₁. The data representing the second factors F₂ may thus be stored jointly or successively to the first factors F₁, that is to say in a same step or in a successive step.

### Second Factors F₂: Medical Histories ANT

Let us consider in this example that the medical history ANT₁, that is to say a disease, is selected from the interface INT₁ from the database of diseases BD_{M}. The information selected and extracted from the database BD_{M} is then associated with the information of said patient P₁. According to an embodiment, in order to associate at least one medical history ANT₁ with a patient P₁, a disease is selected. The fields of description of the disease ANT₁ may be modified when a default value exists or directly defined in the interface INT₁. As an example, the periodicity of occurrence of a sign associated with the selected disease may be entered, just like the duration and the date of the disease. When the medical history ANT₁ is correctly defined, the information may be stored and associated with the identifier of said patient P₁.

According to an embodiment, a plurality of second factors F₂ is defined for a same patient P₁. Thus, a patient may have several medical histories.

According to the invention, the second factors F₂ are thus taken into account in a Bayesian model in order to calculate conditional probabilities of diseases on the basis of the existence or not of this medical history ANT₁.

According to an embodiment, the invention makes it possible of encode a field of a medical history making it possible to exclude the presence of at least one given disease. A property of a medical history ANT₁ may then be “immunizing” of one or more diseases. When the profile of a patient comprises the entering of this medical history ANT₁ comprising said field, the probability associated with the excluded disease is then 0. This property is encoded by a predefined field. The field by default is configured for example on the value “non-immunizing”. The exclusion field may also be encoded when a sign is present in the entering of a profile of a patient. The taking into account of this field makes it possible to generate a probability of 0 for certain diseases excluded by the presence of this sign.

The invention also makes it possible to take into consideration a risk factor linked to the presence of a medical history, more generally a factor F₁, F₂ or F₃, which applies to at least one disease or a group of diseases. The risk factors weight directly the conditional probabilities associated with the diseases.

An interest of considering second factors F₂, corresponding to medical histories is to take into consideration past events of a given patient P₁ in order to calculate a conditional probability of a given disease with the existence of this medical history.

According to another embodiment which is combined with the latter, a medical history is treated jointly with the taking into account of other second factors F₂, that is to say other medical histories when they exist. According to an embodiment, at least one second factor F₂ is correlated with at least one third factor F₃, such as a sign in order to calculate a conditional probability taking into consideration different types of factors F₂, F₃.

To this end, the Bayesian model makes it possible to define quantified logical relationships between the different factors F₁, F₂, F₃ and the diseases making it possible to weight the values of conditional probabilities of diseases in the definition of this Bayesian modelling, the invention makes it possible to treat the interactions between different types of factors F₁, F₂, F₃ in the same way as if the factors were of the same type. Indeed, the Bayesian model of the invention makes it possible to model the logical relationships in the form of conditional probabilities of an event on the occurrence of factors, F₁, F₂, and/or F₃.

The probability P(M |F₁, F₂) is defined corresponding to the probability of having the disease M knowing the presence of the factors F₁ and F₂.

The probability P(F₁, F₂ | M) is defined corresponding to the probability of having the factors F₁ and F₂ knowing the presence of the disease M.

The probability P(F₁, F₂) is defined corresponding to the probability of having the factors F₁ and F₂.

The following relationship is always verified:
P(M | F₁, F₂) = P(F₁,F₂| M) * P(M) / P(F₁,F₂)

The probability of not having the disease is noted P( [symbol] M) and the probability of not having the disease knowing F₁ and F₂ is noted P( [symbol] M |F₁, F₂). One has the following relationships: P( [symbol] M) = 1 - P(M) and P( [symbol] M | F₁, F₂) = 1 - P(M | F₁, F₂).

According to an embodiment of the invention, the factors F₁, F₂ are considered as independent. The following relationship is then obtained:
P(M| F₁,F₂) = P(F₁|M)* P(F₂|M) * P(M) / (P(F₁)*P(F₂))

P(M) corresponds to the probability of having the disease. The method and/or the system of the invention determine as initial prevalence PR₁ or incidence IND₀ value of the disease.

During the determination of the initial value of P(M), the prevalence PR₁ or the incidence IND₁ may take into account a certain number of risk factors or data specific to the profile of the patient. The method and the system of the invention make it possible to compare this information with the data of the database of signs and/or diseases. The method and the system of the invention then make it possible to determine the most relevant prevalence or incidence value in order to calculate the probability P(M). As an example, if a 60 year old patient is a smoker, the prevalence PR₁ that will be determined will take into consideration the probability P(M = lung cancer) of having lung cancer for a given profile with given medical histories and given risk factors.

According to an embodiment, when the prevalence PR₁ is defined in the database of a disease M, the value is determined as the value of P(M). When the value is not defined, the incidence value IN₁ is chosen to determine the initial value of P(M).

According to an embodiment, according to the disease and the profile of the patient {AGE, GEN, GEO, etc.} a priority rule is defined between prevalence and incidence. This rule makes it possible to calculate P(M), that is to say the probability of having the disease with the most relevant information there is. Typically, in a case where prevalence increases with age, incidence may appear to define a more relevant probability P(M) than prevalence for certain patient age ranges.

The specificity and sensitivity values of a set of factors corresponding to the signs are extracted from a database to calculate the probability of a given event, namely the presence of a disease M. The latter values quantify the probabilities of third factors F₃ which are defined as hereafter.

When the specificity of a set of linked factors F₁, F₂ and F₃ for a disease is known and stored in a database, then the probability P*(M=1 |F₁, F₂, F₃) is directly selected in the database when the 3 factors are acquired according to the method and/or the system of the invention. If this joint specificity of these factors is not defined, the system and the method of the invention make it possible to calculate automatically the probability according to the preceding formula while considering the factors as independent.

### Third Factors F₃: The Signs

According to an embodiment, the interface INT₁ or another interface enable the acquisition ACQ₃ of third factors F₃. The third factors F₃ comprise the definition of generic signs Sₖ or detailed signs Sₖᵢ. During the acquisition of data of a new sign S₁, the interface INT₁, for example, makes it possible to extract a sign from the database of signs BDs. The sign S₁ is then called by its ontological concept defining it, that is to say its designation, such as “cough” or “fever”. It involves the generic sign. An interest is to homogenize the signs under a same concept. The generic sign Sₖ or the detailed sign Sₖᵢ is then entered via a plurality of fields making it possible to specify it.

As an example, the characterization of the presence of a sign may be specified by different parameters. According to an example, the frequency of occurrence of a sign may be taken into account and characterized. This characterization may be qualified by a field to determine from among a predefined list of terms such as: {one off, sporadically, regular, frequent, continually}. The frequency of occurrence of a sign may be jointly or alternatively entered by an evolution curve. An example of modification of the occurrence of a sign may be the following: occurrence for 2 to 3 days then disappearance for 6 to 10 days and reoccurrence for 1 to 3 days.

Its frequency, its intensity, is potential anatomical localization visible or “deep” and described or instead a qualification, etc., may be taken into account.

The third factors may thus be defined for each patient by quantifying a certain number of fields making it possible to describe the sign.

FIG. 2 represents an example in which a sign Sₖ forms a common concept with different manifestations of the latter, for example: a “dry cough” or a “wet cough” have for common concept a cough. The set of characteristics common to all the detailed signs Sₖᵢ forms the characteristics of the generic sign Sₖ.

In the example of FIG. 2, three different manifestations S_{K1}, S_{K2}, S_{K3} of the sign S_{K} are represented. These manifestations may concern one or more elements making it possible to define or to specify the sign Sₖ. In FIG. 2, a manifestation Sₖ₁ of the sign Sₖ may be more or less enriched: Sₖ₁(Dₖ₁") represents a more enriched form of the sign Sₖ₁(Dₖ₁') which, itself, represents an enriched form of the sign Sₖ₁(Dₖ₁). Dₖ₁ here represents the description of the sign S_{K1}. It may be for example a more precise description of an evolution of the sign. The invention makes it possible to take into account a specificity value of a detailed sign Sₖᵢ. By default, when it is not entered, the value is identical to the value of the sign of higher level in the ontology of signs. In the case of FIG. 2, the specificity of the sign Sₖ₁(Dₖ₁") is equal to the specificity of the sign Sₖ₁(Dₖ₁') if no value is associated with the latter during its creation.

According to an embodiment, the specificity value may be recalculated from a value of prevalence of diseases and sensitivity of the set of factors.

In FIG. 2, the manifestation of the sign Sₖ₃(Dₖ₃') may provide a more precise description of the sign Sₖ₃(Dₖ₃).

An advantage of better describing a sign S₁ is to increase its specificity SP₁ for a disease. The invention makes it possible to take into account enriched descriptions in order to constitute the most reliable possible database of diseases. Thus, the method of the invention makes it possible to take into account the construction of a database enriched with data of signs BDS.

According to an embodiment, the method of the invention determines from the sensitivity SE₁ and the specificity SP₁ of a sign, a list of conditional probabilities, each being associated with a disease. The taking into account of different medical histories and different signs and the profile of the patient P₁ makes it possible to modify the conditional probabilities P(Mi) associated with each disease Mi of the list that is generated.

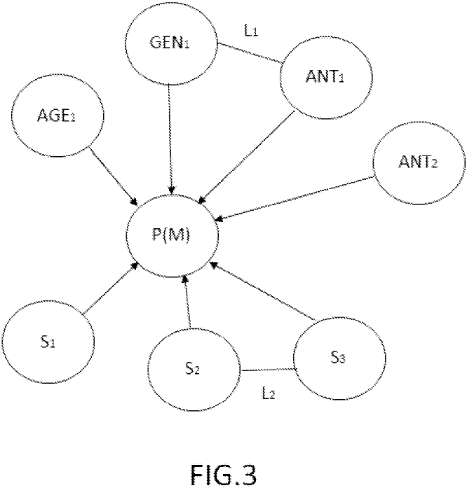

FIG. 3 represents an example of modelling of the Bayesian network making it possible to calculate the probability P(M) of onset of a disease as a function of the presence of first factors F₁ {GEN₁, AGE₁}, second factors F₂ {ANT₁, ANT₂} and third factors F₃ {S₁, S₂, S₃}.

In this embodiment, the representation of the network models a first relationship L₁ between the factors ANT₁ and GEN₁ and a second relationship L₂ between the factors S₂ and S₃.

According to an embodiment, the relationships correspond to a concomitant occurrence of the factors during the presence of the disease.

The factors S₂ and S₃ are linked and make it possible to define the following conditional probability:

According to an embodiment, when the factors GEN₁ and ANT₁ are linked, the Bayesian network model makes it possible to consider the following joint conditional probability: P*(M |F₁, F₂) directly from sensitivity and specificity values of the joint observation. When the factors are not linked, the probability of the disease is then calculated from factors considered as independent.

One then notes P*(M |F₁, F₂): the joint probability defined directly in the database and P(M |F₁, F₂): the probability calculated while considering that the factors F₁ and F₂ are independent.

One of the advantages of the invention is to enable a modelling comprising the joint specificities and sensitivity values of factors being able to be linked. The invention then prioritizes in the calculations the joint values which are defined in the database when they are known.

The system and the method of the invention make it possible to take into account a modelling of an improved naive Bayesian network in which a control of the presence of certain values of probabilities is carried out when factors are capable of being linked. As an example, if a factor F₁ is acquired by a user input and when this factor is linked to a second factor F₂, for example by the presence in the database of a joint probability linked to a defined disease, then the system and the method of the invention make it possible to control the presence of the second factor F₂ in the fields input by a user or to generate automatically an interface in order to obtain from the user information qualifying the presence or not of a second factor F₂.

In this latter embodiment, the linked factors may be linked while being in one of the three sets ENS₁, ENS₂, ENS₃.

The probability of having a disease M is then deduced from the set of data describing the first, second and third factors assumed to be observed or being observed. According to this embodiment, the probabilities of the factors are then deduced from the descriptions and values describing it or defining it. The specificity and sensitivity values serve to calculate the table of probabilities according to the factors considered.

For a given factor, F₁, one considers in this example, a sensitivity value Se of 5 and a specificity value Sp of 4 on a scale of 0 to 6, i.e. in real value after conversion Se=0.875 and Sp = 0.7.

According to this example, we thus have the following probability table:

| | | |
|---|---|---|
| | F₁=0 | F₁=1 |
| M = 0 | P(M=0 \| F₁=0) = (1+ Sp₁) /2 = 0.85 | P(M=0 \| F₁=1) = (1 -Sp₁)/2 = 0.15 |
| M = 1 | P(M=1 \| F₁=0) = 1 - Se₁ = 0.125 | P(M=1 \| F₁=1) = Se₁ = 0.875 |

With:
M = 0: absence of the disease M;
M = 1: presence of the disease M;
F₁ = 0: absence of the factor F₁;
F₁ = 1: presence of the factor F₁.

### Example of Horton’s Disease

The invention makes it possible to describe a disease by designating it and by associating with it a description. In the case of Horton’s disease, the description may indicate a clinical name or a name of general use of the disease such as: the disease is also known by the name “temporal arteritis”. A short description such as the type of disease: “inflammatory disease of the vessels” may be indicated in the database of diseases BDM. Information quantifying the prevalence and the incidence may, moreover, be indicated as: Horton’s disease particularly affects elderly subjects. It is also know by the name “temporal arteritis” due to the fact that one of these arteria (left or right superficial temporal) is affected in the course of the disease.

The information describing the disease may further comprise:
a degree of urgency;
a clinical form of the main table;
a prevalence associated with at least one population comprising a demography, said population being segmented according to a first model;
an incidence associated with at least one population comprising a demography, said population being segmented according to a first model;
a description;
a demographic distribution associated with risk factors.

According to an embodiment of the invention, in a modelling of the Bayesian network, the factors are considered as being independent.

In this example, one considers the following diagnosis:

Diagnostic of the Disease1: Horton’s disease

The prevalence PR₁ of the disease is 1 out of 11,000 in the whole of the population.

The prevalence PR₂ is 10/10,000 above 60 years old. The prevalence PR₂ is selected automatically in the database when the age of the individual is greater than 60 years. In this situation, the system and/or the method of the invention determine automatically the value stored in the database of linked factors. Here age is considered as a risk factor increasing the probability of having the disease. This factor is linked to the initial prevalence PR₁ or P(M).

The disease is distributed with a Women/Men ratio of ⅔ - ⅓.

A patient P₁ manifests the following signs (Factors F₃):
Sign 1 = Morning headaches
Sign 2 = Isolated fever: Specificity (Sp1): <5% for Horton’s disease / Sensitivity (Se1): 90% in Horton’s disease;
Sign 3 = Isolated fatigue: Specificity (Sp2): <5% for Horton’s disease / Sensitivity (Se2): 60% in Horton’s disease;
Sign 4 = Loss of weight without other cause: Specificity (Sp3): 15% for Horton’s disease / Sensitivity (Se3): 50% in Horton’s disease;
Sign 5 = Pain with palpation of the temporal artery (on the side of the headache): Specificity (Sp4): 75% for Horton’s disease / Sensitivity (Se4): 40% in Horton’s disease.

The profile of the Patient P₁ is considered of which the factors F₁ comprise the age AGE₁ = 72 years, the gender GEN₁ = woman and a geographic information GEO₁ = Paris, France. The second factors F₂ comprise a medical history ANT₁.

The method generates a list of diseases with associated probabilities, List 1:
Horton’s disease: Proba1 %
Disease 2: Proba2 %
Disease 3: Proba3 %
Disease 4: Proba4 %
Disease 5: Proba5 %

The probability P(M) with M being Horton’s disease may be written as a function “f” of different parameters and variables:

P(M | {Fi}iε[1 ;N]) = f(ANT₁, Sp₁, Sp₂, Sp₃, Sp₄, Se₁, Se₂, Se₃, Se₄, (PR₁ or PR₂ or IN₁))

One defines P(S=0 |M=0) the probability that the event “S” does not occur knowing that the disease is not present.

$$\text{P} \left(\text{S=0\textbar M} \text{=0}\right) = \left(\text{1+} {\text{Sp}}_{\text{1}}\right) \text{/} \text{2}$$

$$\text{P} \left(\text{S=1\textbar M=0}\right) = \left(\text{1} - {\text{Sp}}_{\text{1}}\right) \text{/2}$$

$$\text{P} \left(\text{S=1\textbar M=} \text{1}\right) = \text{Se}$$

$$\text{P} \left(\text{S=0\textbar M=1}\right) = \text{1} - {\text{Se}}_{\text{1}}$$

For each sign Sᵢ and medical history ANTᵢ, the table of these 4 probabilities that serve to supply each node of the model is calculated; the joint distribution is calculated from these probability tables.

These four relationships are thus written for each specificity Spᵢ and sensitivity Seᵢ value of each sign Si.

### Taking in Account Rare Diseases

According to an embodiment, the interface of the system of the invention makes it possible to configure the number of diseases associated with a probability which is displayed in a list LIST₁. By default, this value may be defined at 5.

According to an embodiment, a tab making it possible to activate or to deactivate the taking into account of rare diseases is present on the interface. Thus, a user may decide to display diseases associated with low probability on account of their rarity. This option notably makes it possible to verify that a set of signs and the profile of a patient may be associated with a rare disease.

Another advantage is to enable a user of take a suitable action if the indices reinforce the possibility of a disease having low probability in the patient. For example, a suitable action could be to recommend complementary tests to the patient in order to differentiate the presence of a rare disease.

### Access Interface

According to an embodiment, the list LIST₁ of diseases that is generated by the system or the method of the invention generates a set of icons making it possible to access a file associated with the disease selected in the list LIST₁. The file makes it possible to present to a user the set of characteristics of the disease that is entered in the database.

In particular, the file makes it possible to display the specificity and sensitivity values of the signs present in the disease.

Thus, the user may analyze the data of each sign individually.

### Pharmacovigilance

According to an exemplary embodiment, the method of the invention makes it possible to take into account fourth factors F₄ in the calculation of conditional probabilities associated with diseases generated in a list LIST₁. The fourth factors F₄ are defined during a fourth acquisition of information ACQ₄. The fourth acquisition ACQ₄ of information comprises the name of an active principle and at least the presence or not of a known effect.

The system of the invention comprises a database of active principles which may be associated or not with products such as medications. A memory makes it possible to save the probability that a sign is linked to the taking of a medication or that a disease is linked to the taking of a medication. The probability is then predefined for a given population.

The interface of the system of the invention makes it possible to take into account the taking of medication by at least one patient. According to an embodiment, the duration and the dates of the treatment and the posology may be entered in the interface. The data are then stored and associated with a given patient.

The calculation of the probabilities of diseases of the list LIST₁ is then weighted by the probabilities that a sign, a grouping of signs or a disease is linked to the taking of a medication according to the properties entered namely: name of the product, duration and dates of the treatment, posology, etc.

According to another aspect, the invention makes it possible to take into account the effects resulting from the taking of an active principle, such as a medication, to deduce therefrom the causal relationships with the onset of effects or not in one or several patients.

This embodiment makes it possible to dissociate the effects arising from the taking of a first active principle which produce at least one identified pharmacological effect from a second active principle or a placebo that does not produce this pharmacological effect.

FIG. 1 introduces the acquisition of information of the fourth acquisition ACQ₄ as an event EVN₁.

An interest of this embodiment is to carry out tests on two sets of patients, ENS_{MED} and ENS_{PBO}, of which a set ENS_{MED} comprises the patients having received a medication and a set ENS_{PBO} comprises the patients having received the placebo. The tests aim to quantify the incidences of the signs, affections emerging and undesirable, validated by clinical research investigator physicians for each of the two sets, to evaluate therefrom the differences in statistically significant incidences and to evaluate therefrom the potential imputability to a medication on the occurrence of a sign Sₖ or of a disease Mᵢ in a patient in general.

The method is then repeated a plurality of times for each patient of the two sets. The values may be for example averaged over the set of patients of a same set. According to another embodiment, other functions may be used such as a median function. The averaged conditional probabilities of each disease of each aggregation of list according to the sets considered are then compared. The method of the invention then makes it possible to generate automatically at least one indicator plotting a difference between two averaged probabilities associated with a same disease in each aggregated list of each group. An advantage is to measure the significant differences making it possible to isolate effects linked to the taking of a medication for a group of patients given that it does not produce the same effect in the patients of the set ENS_{PBO}.

According to an embodiment, the fourth acquisition of data ACQ₄ comprises a description of a set of signs Sₖ associated with the taking of a medication. In this respect, the data of these signs Sₖ form part of the set of data ENS_{MED}. The signs Sₖ are then taken into account in the calculation of the conditional probabilities of the list LIST₁. Here, the sensitivity SEₖ and specificity SPₖ values relative to a set of diseases are integrated in the model. An interest is to measure the influence of the taking of a medication in the occurrence of a disease. In other words, it is possible of decrease diagnostic errors by isolating the effects arising from the taking of medication.

According to an embodiment, the invention generates:
a first list LIST₁ comprising the conditional probabilities taking into account the signs associated with the medication administered by a patient P₁ and;
a second list LIST₂ comprising the conditional probabilities not taking account of the signs associated with the medication taken by a patient P₁.

A user may then deduce directly, if it exists, the effect of a medication in the occurrence of an identified disease.

1. A system comprising:
a calculator comprising one or more processors and a non-transitory computer readable medium storing computer program instructions that when executed cause the one or more processors to generate a probability associated with a respective disease in a list of diseases based on input factors and a Bayesian network model, the Bayesian network model being stored in a memory and encoding nodes and relationships between respective nodes corresponding to the input factors, wherein:
each node is associated with a given factor,
at least some of the nodes have a relationship that pairs the node with at least one other node, and
each relationship pairing two nodes indicates at least one probability associated with a respective disease in the list of diseases that corresponds to the factors of the different nodes in the pairing;
an interface of the calculator configured to receive:
a first acquisition of a first set of first factors in relation to a first patient and storing said first factors in a memory, said first factors comprising at least:
an age value, and
a gender value;
a second acquisition of a second set of second factors indicative of a risk factor of at least one disease and information specific to said patient and storing said second factors in a memory, said at least one disease being extracted from a database, each disease being associated with a first prevalence statistic and/or a first incidence statistic, and each disease being associated with a list of signs, at least one sign corresponding to a symptom of a disease;
a third acquisition of a third set of third factors describing at least one sign and storing said third factors in a memory of the system, each sign being associated with a first sensitivity statistic and a first specificity statistic for each disease of a predefined list of diseases associated with the sign, wherein:
the first sensitivity statistic is encoded according to a first predefined scale of values corresponding to respective ranges of values of a first predefined distribution of the first sensitivity statistic of the disease, the maximum number of values of the first scale of values being less than or equal to 11, and
the first specificity statistic is encoded according to a second predefined scale of values corresponding to respective ranges of values of a predefined distribution of the first specificity statistic, the maximum number of values of the second scale of values being less than or equal to 11,
wherein obtaining a set of third factors comprises:
determining that first received user input corresponds to a generic descriptor of a given sign in an ontology of signs,
obtaining, based on the ontology of signs and the given sign, descriptors of detailed signs to which the given sign is generic in the ontology of signs,
providing, for selection via the interface, the descriptors of the detailed signs, and
obtaining a descriptor of a detailed sign based on second received user input, the descriptor of the detailed sign being associated with at least one of a second sensitivity statistic or second specificity statistic of the detailed sign that differs from that of the given sign for at least one disease associated with the given sign;
said calculator generating, based on the Bayesian network model and input data comprising the data received by the first, second and third acquisitions, an output comprising a set of probabilities, each probability being associated with a given disease of the list and generated based on nodes corresponding to factors represented in the input data and probabilities associated with the given disease for pairings of the nodes, wherein at least one node corresponds to the given sign and the probability of the at least one disease is based on said at least one of the second sensitivity statistic or the second specificity statistic of the detailed sign; and a graphical interface for displaying said probabilities in the set of probabilities in association with respective diseases of the list.

2. The system according to claim 1, wherein the interface of the calculator is further configured to receive a fourth acquisition of fourth factors describing a given medical product and at least one second sign associated with said given medical product, said second sign being associated with a third sensitivity statistic and a third specificity statistic for each disease of a second predefined list of diseases associated with said second sign; the calculator generating the set of probabilities while taking into account as input the data of the fourth acquisition to generate the first list.

3. The system according to claim 2, comprising a memory for saving data describing at least two medical products and saving data coming from a plurality of data acquisitions derived from a set of patients in such a way that a first group of patients is associated with the first product and a second group of patients is associated with the second product, the calculator generating two lists of conditional probabilities, each probability being associated with a given disease, and detecting, by the calculator based on a comparing of the two lists, at least one difference in probabilities for a same disease between the two lists when said difference is above a predefined threshold.

4. The system according to claim 1, comprising a linguistic resource comprising an ontology, a dictionary or a synonyms corpus, wherein signs in the database are detected based on associations formed between descriptors of signs or diseases with user input terms.

5. The system according to claim 1, wherein each third factor comprises a plurality of properties describing a sign and stored in a memory of the system, each property being associated with a sensitivity and specificity value.

6. The system according to claim 1, wherein the Bayesian network model comprises a modelling of at least one relationship between two factors of one of the four sets of factors, said modelling specifying if the factors are independent or dependent and being stored in a memory of the system.

7. The system according to claim 6, wherein:
when no dependency relationship between at least two factors present in the acquired data is specified, the calculation of a probability of a disease comprises a calculation of conditional probability from factors considered as independent;
when said Bayesian modelling specifies a dependency relationship between at least two factors, the calculation of a probability of a disease comprises a selection in the database of one or more of a conditional probability or a selection of a sensitivity and/or specificity value of the factors considered jointly.

8. The system according to claim 1, wherein the second sensitivity statistic is encoded according to a third predefined scale of values and the second specificity statistic is encoded according to a fourth predefined scale of values.

9. The system according to claim 1, wherein at least one of the first predefined scale of values and of the second predefined scale of values comprises a number of values comprised between 5 and 8.

10. A computer-implemented method, the method comprising:
generating, with one or more processors, a Bayesian network model configured to output a probability associated with a respective disease in a list of diseases based on input factors, the Bayesian network model encoding nodes and relationships between respective node pairings corresponding to the input factors, wherein generating the Bayesian network model comprises:
encoding, by one or more processors, each node with a given factor, and
encoding, by one or more processors, for each of at least some of the nodes, a relationship that pairs the node with at least one other node based on at least one probability associated with a respective disease in the list of diseases that corresponds to the factors of the different nodes of the pairing;
obtaining in a first acquisition, with one or more processors, in relation to a first patient, a first set of first factors comprising:
an age value, and
a gender value;
obtaining in a second acquisition, with one or more processors, a second set of second factors indicative of a risk factor of at least one disease and information specific to said patient, said disease being extracted from a database, each disease being associated with a first prevalence statistic and a first incidence statistic, and each disease being associated with a list of signs;
obtaining in a third acquisition, with one or more processors, a third set of third factors describing at least one sign, each sign being associated with a first sensitivity statistic and a first specificity statistic for each disease of a predefined list of diseases associated with the sign, wherein:
the first sensitivity statistic is encoded according to a first predefined scale of values corresponding to respective ranges of values of a first predefined distribution of the first sensitivity statistic of the disease, the maximum number of values of the first scale of values being less than or equal to 11, and
the first specificity statistic is encoded according to a second predefined scale of values corresponding to respective ranges of values of a predefined distribution of the first specificity statistic, the maximum number of values of the second scale of values being less than or equal to 11,
wherein obtaining a set of third factors comprises:
determining that first received user input via an interface corresponds to a generic descriptor of a given sign in an ontology of signs,
obtaining, based on the ontology of signs and the given sign, descriptors of detailed signs to which the given sign is generic in the ontology of signs,
providing, for selection via the interface, the descriptors of the detailed signs, and
obtaining, a descriptor of a detailed sign based on second received user input, the descriptor of the detailed sign being associated with at least one of a second sensitivity statistic or second specificity statistic of the detailed sign that differs from that of the given sign for at least one disease associated with the given sign; and
determining, based on the Bayesian network model and input data comprising the initial probabilities and the data of the first, second and third sets of factors, an output comprising a set of probabilities, each probability associated with a given disease of the list and generated based on nodes corresponding to factors represented in the input data and probabilities associated with the given disease for pairings of the nodes, wherein at least one node corresponds to the given sign and the probability of the at least one disease is based on said at least one of the second sensitivity statistic or the second specificity statistic of the detailed sign.

11. The method according to claim 10, wherein the probability associated with a disease of the list is a conditional probability of a disease with the occurrence of a set of factors comprising at least three elements selected from:
a predefined sign;
a predefined medical history;
a risk factor;
a predefined age;
a predefined gender; and
a predefined geographic location.

12. The method according to claim 10, wherein at least one factor of the second data acquisition is associated with a medical history and comprises at least one of the following criteria:
a first date of appearance;
a frequency of appearance; and
a genetic information.

13. The method according to claim 10, wherein the Bayesian network model comprises, for at least one node of the network, a modelling of at least one relationship between two factors of one of the four sets of factors, said modelling specifying if the factors are independent or dependent, the dependency relationship between at least two factors being modelled by a value of joint conditional probability of a disease knowing the factors present.

14. The method according to claim 10, further comprising:
verifying the existence of a relationship between factors acquired from the different sets in the database of signs or diseases; and
selecting the value of the joint probability associated with the linked factors.

15. The method according to claim 10, comprising a fourth acquisition of fourth factors describing a given medical product and at least one second sign associated with said given medical product, said second sign being associated with a third sensitivity statistic and a third specificity statistic for each disease of a second predefined list of diseases associated with said second sign; wherein the step of generating the set of probabilities comprises taking into account as input the data of the fourth acquisition.

16. The method according to claim 15, further comprising:
a plurality of data acquisitions for respective patients in a plurality of patients, each patient being associated with a first product or with a second product, a first group of patients being associated with the first product and a second group of patients being associated with the second product, wherein the generation step comprises:
generation of two lists of conditional probabilities, each probability being associated with a given disease, and
determining the presence of at least one difference in probabilities associated with a same disease and for which the difference is above a predefined threshold based on a comparison of the two lists.

17. The method according to claim 10, wherein the sensitivity or the specificity of a sign of an input of the database comprises:
either a value expressing a probability;
or a value selected from a predefined discrete scale, said predefined discrete scale associating with each of its values a probability by age and/or gender group.

18. The method according to claim 10, wherein the third acquisition is carried out by means of an interface in which the selection of a given sign automatically leads to the generation of a first selection of one or more associated signs, said selected signs being associated with the given sign in at least one disease, said interface comprising a menu displaying said selection of signs.

19. The method according to claim 10, wherein the third acquisition leads to the generation of a second selection of tests, said tests comprising a description aiming to identify the presence of at least one sign in the patient.

20. The method according to claim 10, further comprising a graphic interface and comprising the display of said list within the graphic interface.

21. A computer readable medium, comprising a program including software code portions for the execution of the steps of the method according to claim 10 when said program is executed on a computer.
